(19) United States
(12) Patent Application Publication
(10) Pub. No.: US 2010/0331600 A1
(43) Pub. Date: Dec. 30, 2010

(54) SYSTEM FOR DISPENSING RADIO-PHARMACEUTICALS AND MEASURING RADIATION DOSAGE OF IT

(75) Inventors: Won-gyu Bae, Gyeonggi-do (KR); Myeong-sik Lee, Gyeonggi-do (KR)

Correspondence Address: LOWE HAUPTMAN HAM & BERNER, LLP, 1700 DIAGONAL ROAD, SUITE 300, ALEXANDRIA, VA, 22314, US

(73) Assignees: Hyun-mi PARK, Guri-si, Gyeonggi-do (KR); Myeong-sik LEE, Yongin-si Gyeonggi-do (KR)

(21) Appl. No.: 12/919,610

(22) PCT Filed: Dec. 31, 2008
(86) PCT No.: PCT/KR2008/007818

§ 371 Date: Aug. 26, 2010

(30) Foreign Application Priority Data
Feb. 29, 2008 (KR) 10-2008-0019394
Jul. 16, 2008 (KR) 10-2008-0069247

(51) Int. Cl. A61M 36/06

(52) U.S. Cl. 600/5

(57) ABSTRACT

The present invention provides a system for dispensing radiopharmaceuticals and measuring a radiation dosage thereof. In the system, the operation of dispensing a predetermined dose of radiopharmaceuticals from a vial (21) to an syringe (31) and the operation of measuring a radiation dosage of the radiopharmaceuticals dispensed to the syringe can be sequentially conducted without a risk of radiation leaking. Therefore, the system can markedly reduce the frequency of radiation exposure and the radiation exposure dose. Furthermore, when the syringe is moved downwards from a radiopharmaceutical dispensing apparatus (101) into a radiation dosage measuring apparatus (141) to measure a radiation dosage, the filling opening of the vial is displaced from a position aligned with the syringe, thus preventing radiopharmaceuticals remaining in the vial from affecting the measurement of the radiation dosage of the syringe.

### TECHNICAL FIELD

The present invention relates, in general, to systems for dispensing radiopharmaceuticals and measuring the radiation dosage thereof and, more particularly, to a system for dispensing radiopharmaceuticals and measuring a radiation dosage thereof which can sequentially conduct, in an internal space, the operation of dispensing a predetermined dose of radiopharmaceuticals to be used for treatment or examination from a vial to an syringe and the operation of measuring a radiation dosage of the radiopharmaceuticals dispensed to the syringe.

### BACKGROUND ART

Early detection is of vital importance for successful treatment of cancer. Positron Emission Tomography (hereinafter, referred to as “PET”) using radioactive isotopes has been widely used as a representative method for the early detection of cancer.

In the PET test, radiopharmaceuticals (liquid solutions), for example, 2-[F-18] fluoro-2-deoxy-D-glucose, is administered to a patient's body through intravenous injection. Radiation emitted from specially designated tissue or organs to be examined is image-processed.

In the case where the PET test is used, fine cancer cells or tumor tissue of several millimeters which cannot be detected by the prior test can be detected in the early stages. As well, while treatment is being administered, whether cancer has spread or a second attack has appeared, or effect of an administered anticancer drug can be effectively checked.

A nuclide having a short half-life is used as a radiopharmaceutical in consideration of the difficulty of transporting and storage or problems of contamination and radiation exposure to the human body. When necessary, a unit dose of radiopharmaceuticals contained in a vial is dispensed to an syringe. Here, depending on the half-life of the radiopharmaceutical, a required radiation dosage is varied, and a dose of radiopharmaceuticals is also varied in response to the required radiation dosage. Hence, for precise medical examination, a radiation dosage of a radiopharmaceutical dispensed to the syringe must be previously measured.

Because the radiopharmaceuticals are friendly to special organs or diseases and have a short half-life, even though the radiopharmaceuticals are administered to a patient, the radiation exposure dose is negligibly small. However, in terms of an operator who must always handle radiopharmaceuticals, there is a problem in that the radiation exposure dose is increased while the work of dispensing radiopharmaceuticals and measuring a radiation dose thereof is repeated on a daily basis.

In an effort to overcome the above problems, a PET dispensing system provided with a radiation shield made of lead glass for preventing radiation leakage has been used to prevent or minimize internal radiation exposure to the operator attributable to evaporation of isotopes or external radiation exposure to the operator attributable to direct exposure during the process of dispensing radiopharmaceuticals. Furthermore, even when the syringe is moved to measure a radiation dose of the radiopharmaceuticals dispensed to the syringe, a separate radiation shield has been used to minimize radiation exposure.

However, in this case, there are disadvantages in that each and every time the operator must move and place radiopharmaceuticals into a system and measure a radiation dosage of the set radiopharmaceuticals. In addition, the number of times that the operator is exposed to radiation during the above process, and the radiation exposure dose, are only slightly reduced. In particular, radiation exposure to the hands of the operator may seriously menace the health of the operator.

### DISCLOSURE OF INVENTION

### Technical Problem

Accordingly, the present invention has been made keeping in mind the above problems occurring in the prior art, and an object of the present invention is to provide a system for dispensing radiopharmaceuticals and measuring a radiation dosage thereof which, without a risk of radiation leakage to the outside, can sequentially conduct therein the operation of dispensing a predetermined dose of radiopharmaceuticals from a vial to an syringe and the operation of measuring a radiation dosage of the radiopharmaceuticals dispensed to the syringe, thus being more convenient for an operator, and markedly reducing the frequency of radiation exposure and the radiation exposure dose.

### Technical Solution

In order to accomplish the above object, the present invention provides a system for dispensing radiopharmaceuticals and measuring a radiation dosage thereof, including: a radiopharmaceutical dispensing apparatus to dispense a predetermined dose of radiopharmaceuticals from a vial to an syringe, the vial being received in a first radiation shield and containing radiopharmaceuticals, the syringe being received in a second radiation shield; and a radiation dosage measuring apparatus provided under the radiopharmaceutical dispensing apparatus to measure a radiation dosage of the radiopharmaceuticals dispensed to the syringe which is moved downwards from the radiopharmaceutical dispensing apparatus, wherein when the radiation dosage measuring apparatus measures the radiation dosage of the radiopharmaceuticals in the syringe, a filling opening of the vial is disposed at a displaced position to prevent the radiation dosage measurement from being affected by radiopharmaceuticals remaining in the vial after the predetermined dose of radiopharmaceuticals is dispensed to the syringe.

Preferably, the radiopharmaceutical dispensing apparatus may include: an syringe holder to support the syringe such that an injection needle of the syringe is oriented upwards; a bottle casing drive unit provided above the syringe holder to drive a vial receiving casing containing the vial therein; a preset dose dispensing unit to dispense a preset dose of radiopharmaceuticals from the vial to the syringe by moving a piston of the syringe upwards or downwards in a state in which the injection needle of the syringe is stuck into the filling opening of the vial; an syringe lift unit to move, upwards or downwards, the syringe holder supporting the syringe, into which the preset dose of radiopharmaceuticals is injected by the preset dose dispensing unit; and a control unit to control the bottle casing drive unit such that when the vial receiving casing is moved downwards while the vial receiving casing is oriented such that the filling opening of the vial faces downwards, the injection needle of the syringe supported by the syringe holder is stuck into the filling opening of the vial and the preset dose dispensing unit holds the piston of the syringe supported by the syringe holder, the control unit controlling the bottle casing drive unit such that when the vial receiving casing is moved upwards, the injection needle of the syringe is removed from the vial and the preset dose dispensing unit releases the piston of the syringe, and then the vial receiving casing is moved such that the filling opening of the vial is oriented upwards, the control unit controlling the preset dose dispensing unit to move the piston of the syringe held by the preset dose dispensing unit upwards or downwards, the control unit controlling the syringe lift unit to move the syringe containing the preset dose of radiopharmaceuticals upwards or downwards.

Alternatively, the radiopharmaceutical dispensing apparatus may include: an syringe holder to support the syringe such that an injection needle of the syringe is oriented upwards; a bottle casing drive unit provided above the syringe holder to drive a vial receiving casing containing the vial therein; a preset dose dispensing unit to dispense a preset dose of radiopharmaceuticals from the vial to the syringe by moving a piston of the syringe upwards or downwards in a state in which the injection needle of the syringe is stuck into the filling opening of the vial; an syringe lift unit to move, upwards or downwards, the syringe holder supporting the syringe, into which the preset dose of radiopharmaceuticals is injected by the preset dose dispensing unit; and a control unit to control the bottle casing drive unit such that when the vial receiving casing is moved downwards while the vial receiving casing is oriented such that the filling opening of the vial faces downwards, the injection needle of the syringe supported by the syringe holder is stuck into the filling opening of the vial and the preset dose dispensing unit holds the piston of the syringe supported by the syringe holder, the control unit controlling the bottle casing drive unit such that when the vial receiving casing is moved upwards, the injection needle of the syringe is removed from the vial and the preset dose dispensing unit releases the piston of the syringe, and then the vial receiving casing is moved leftwards or rightwards, the control unit controlling the preset dose dispensing unit to move the piston of the syringe held by the preset dose dispensing unit upwards or downwards, the control unit controlling the syringe lift unit to move the syringe containing the preset dose of radiopharmaceuticals upwards or downwards.

### ADVANTAGEOUS EFFECTS

In the system for dispensing radiopharmaceuticals and measuring a radiation dosage thereof according to the present invention, the operation of dispensing a necessary dose of radiopharmaceuticals from a vial to an syringe and the operation of measuring a radiation dosage of the radiopharmaceuticals contained in the syringe without being affected by the radiopharmaceuticals remaining in the vial can be sequentially conducted in a single system by external manipulation. Therefore, it becomes unnecessary for an operator to dispense radiopharmaceuticals to every syringe one by one or measure a radiation dosage every time the dispensing operation is conducted, thus facilitating the work, and making it proceed smoothly. Furthermore, the entire operation is conducted inside the system without a risk of radiation leakage, so that the frequency with which the operator is exposed to radiation and the time of radiation exposure are markedly reduced, thus preventing or minimizing a risk of radiation exposure.

In particular, in the case where the system according to the present invention is constructed such that an entire vial receiving casing can be moved upwards, downwards, leftwards and rightwards but the vial is not moved with respect to the vial receiving casing, an injection needle of the syringe can be exactly stuck into a filling opening of the vial in the process of dispensing a necessary dose of radiopharmaceuticals from the vial to the syringe. Moreover, the system of this case can easily and reliably prevent radiopharmaceuticals remaining in the vial from affecting measurement of a radiation dose of radiopharmaceuticals contained in the syringe.

### BRIEF DESCRIPTION OF DRAWINGS

FIG. 1 is a perspective view of a system for dispensing radiopharmaceuticals and measuring radiation dosage thereof, according to a first embodiment of the present invention;

FIGS. 2 and 3 are views showing the system of FIG. 1 to illustrate a process of inserting a vial containing radiopharmaceuticals into a vial receiving casing and inserting an syringe, into which the radiopharmaceuticals are injected, into a radiation shield;

FIGS. 4 through 6 illustrate the system of FIG. 1 to show a process of dispensing a necessary dose of radiopharmaceuticals contained in the vial to the syringe;

FIGS. 7 and 8 illustrate the operation mechanism of a process of coupling a preset dose dispensing unit to a piston of the syringe during the process of FIGS. 4 and 5;

FIGS. 9 through 13 are views illustrating a process of measuring a radiation dosage of radiopharmaceuticals injected into the syringe of the system of FIG. 1;

FIG. 14 is a perspective view of a system for dispensing radiopharmaceuticals and measuring a radiation dosage thereof, according to a second embodiment of the present invention;

FIG. 15 is a view showing the operation of the system of FIG. 14, in which a necessary dose of radiopharmaceuticals is set, and the set dose of radiopharmaceuticals contained in a vial is automatically dispensed to an syringe; and

FIGS. 16 through 19 are views showing the system of FIG. 14 to illustrate movement of a vial receiving casing, when measuring radiation dosage of radiopharmaceuticals injected into the syringe, and the operation of elements pertaining to the movement of the vial receiving casing.

### DESCRIPTION OF THE ELEMENTS IN THE DRAWINGS

11: syringe insert door 12: shielding wall
13: control box 15: cabinet
16: grip 17: wheel
21: vial
31: syringe 32: cylinder
33: piston 34, 34′: radiation shield
100: system for dispensing radiopharmaceuticals and measuring radiation dosage thereof according to first embodiment
101: radiopharmaceutical dispensing apparatus
102: radiation shield support
103: seating hole 104: syringe holder
111: vial receiving casing 112, 131: frame
113: bottle casing connection rod 114: connection box
115: casing lift motor
116: casing lift force transmission means 117: casing rotating motor
118: casing rotating force transmission means
121: piston coupling protrusion
122: protrusion pushing force transmission means
124: spring 125: piston guide
126: piston vertical moving member 127: injection dose control handle
128: handle rotating force transmission means
129: graduated ruler 130: digital graduation indicator
132: syringe lift motor
133: syringe lift force transmission means 134: slide rail
136: syringe holder lift unit 137: connection member
141: radiation dosage measuring apparatus
100′: system for dispensing radiopharmaceuticals and measuring radiation dosage thereof according to first embodiment
102′: radiation shield support 103′: seating hole
111′: vial receiving casing 111-1′: casing receptacle
112′: frame 114′: connection box
115′: casing lift motor
116′: casing lift force transmission means
117′: casing lateral drive motor
118′: casing lateral drive force transmission means
127′: injection dose control motor
128′: dose control force transmission means

### BEST MODE FOR CARRYING OUT THE INVENTION

Hereinafter, preferred embodiments of the present invention will be described in detail with reference to the attached drawings. The preferred embodiments are only examples proposed to illustrate the present invention in detail such that those skilled in the art can easily implement the present invention, and they must not be regarded as limiting the scope and spirit of the present invention.

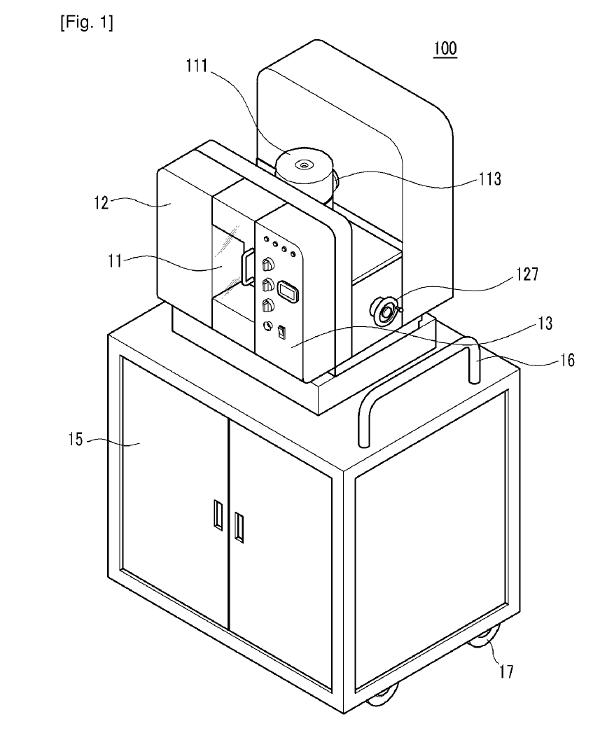

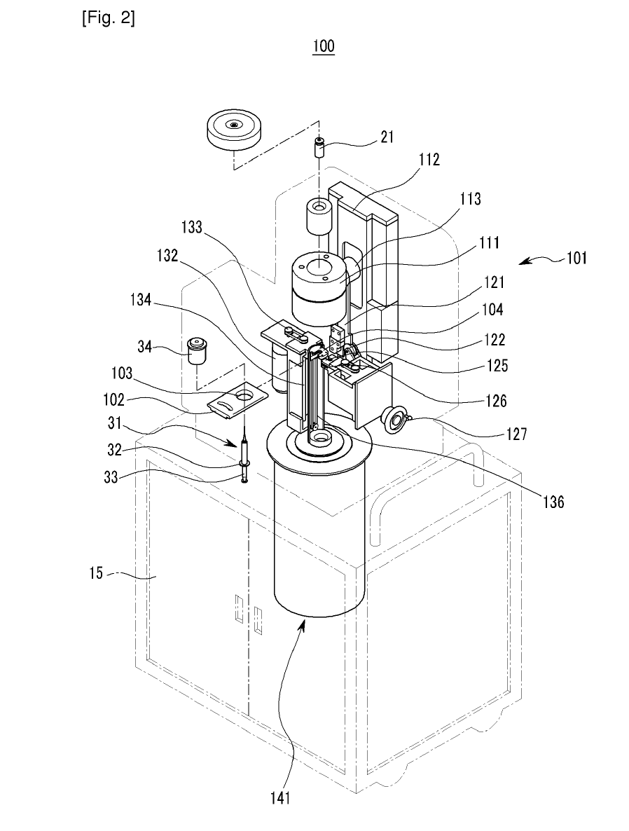

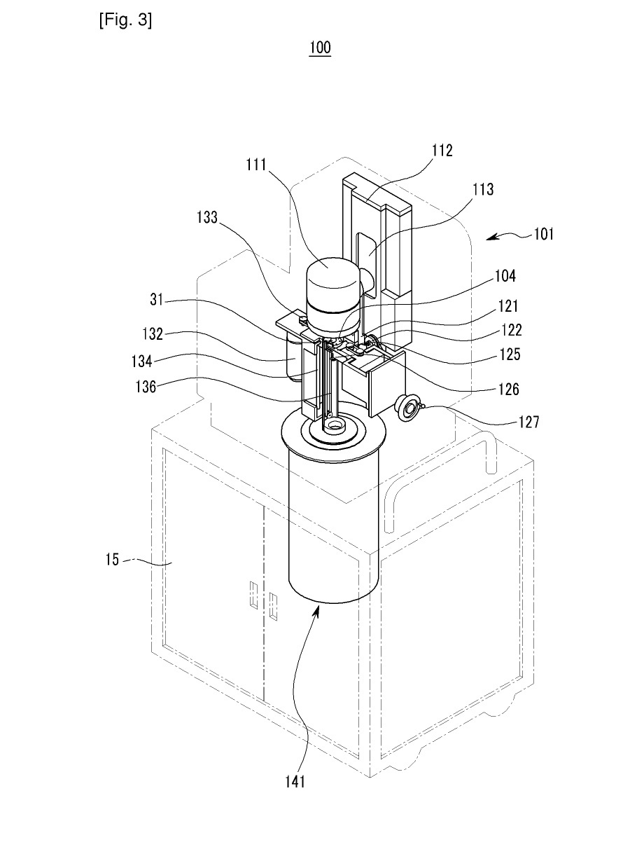

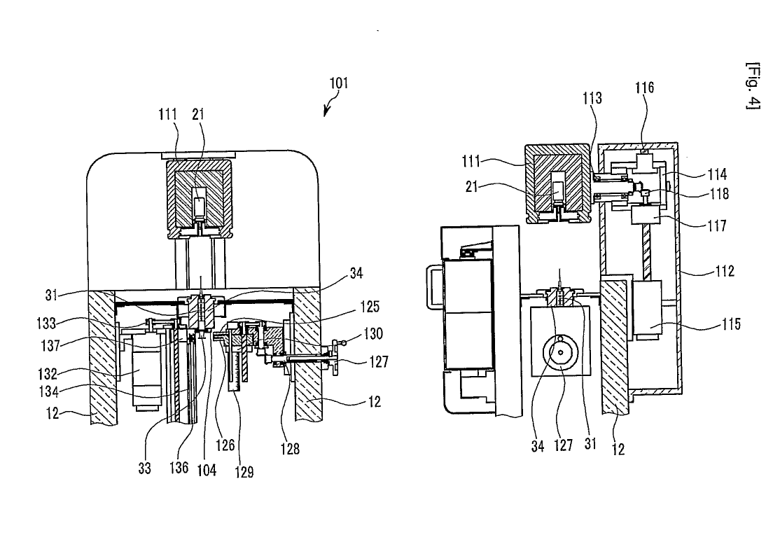

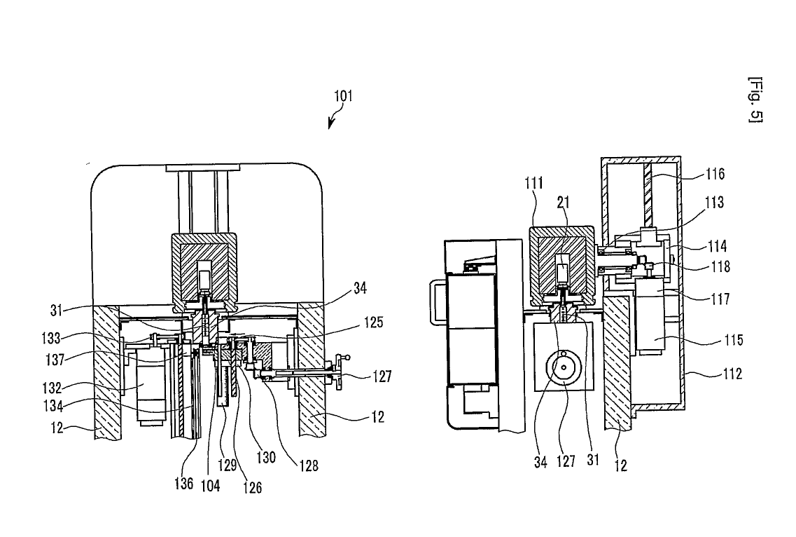

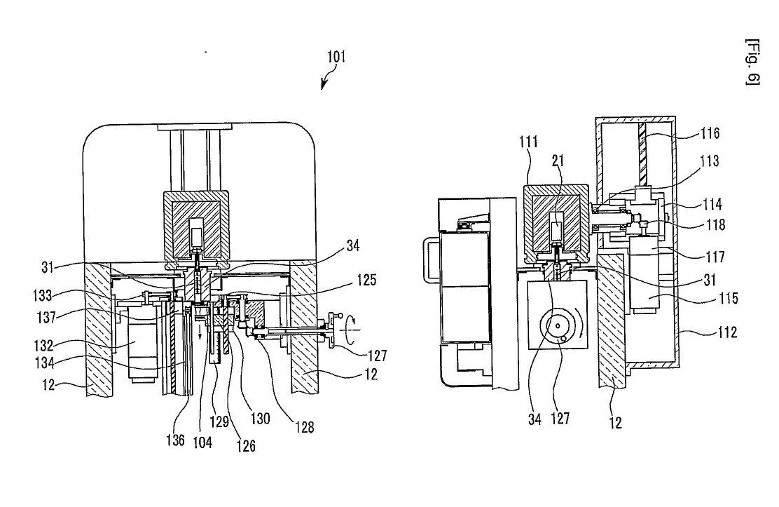

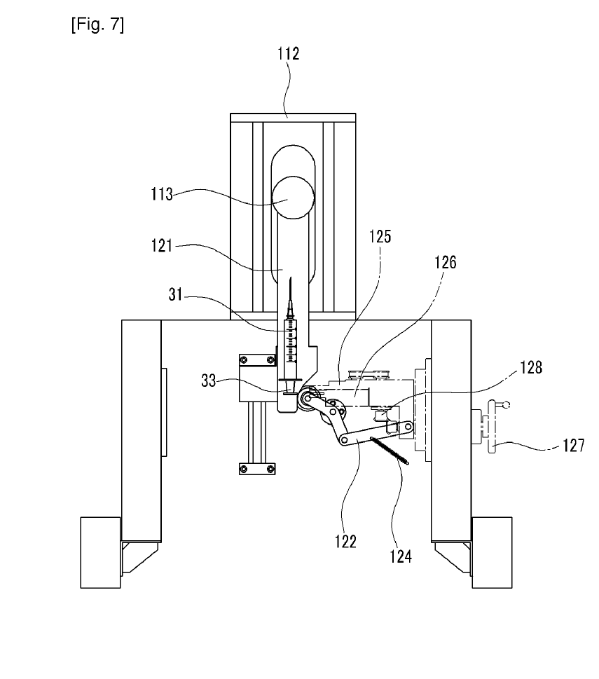

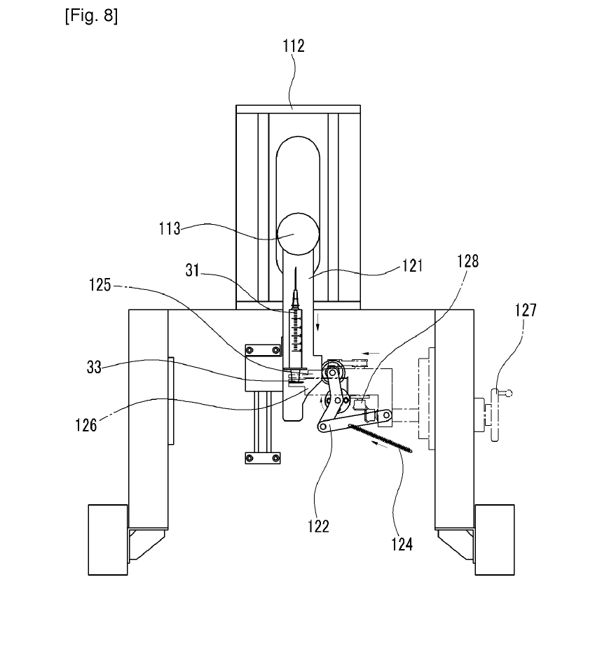

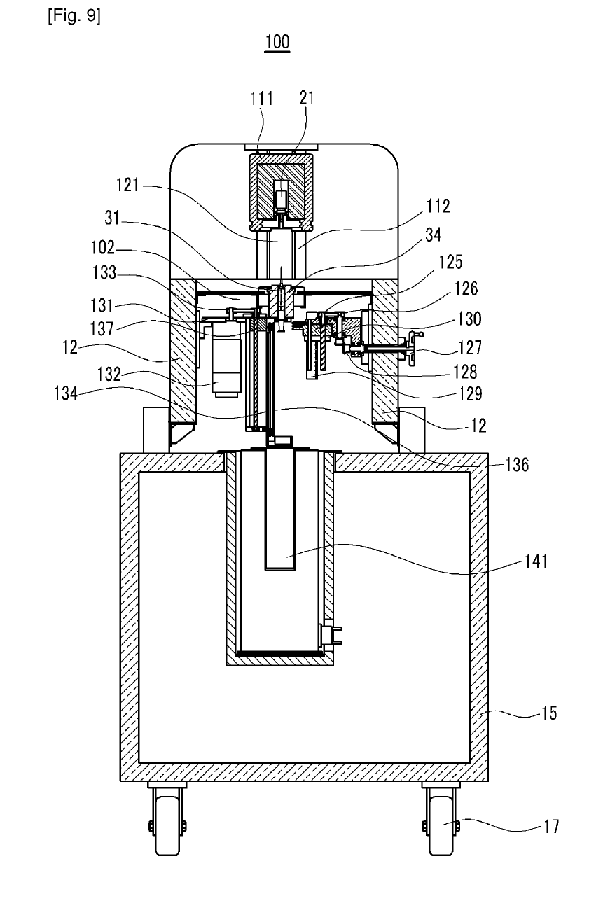

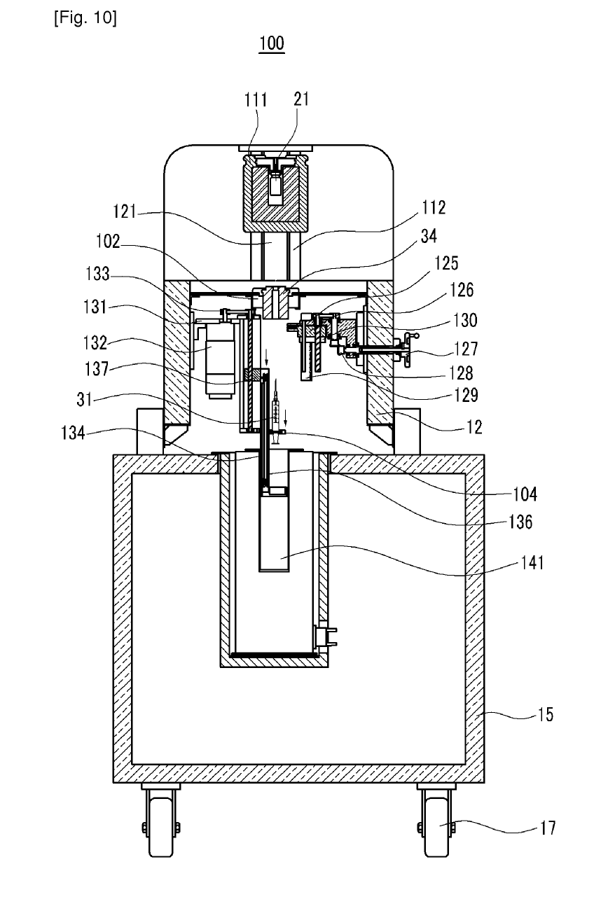

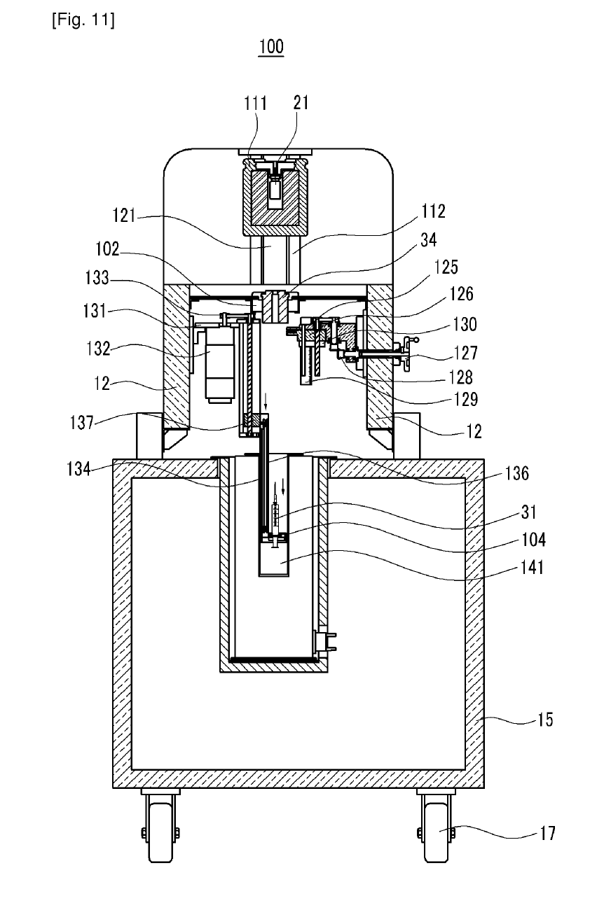

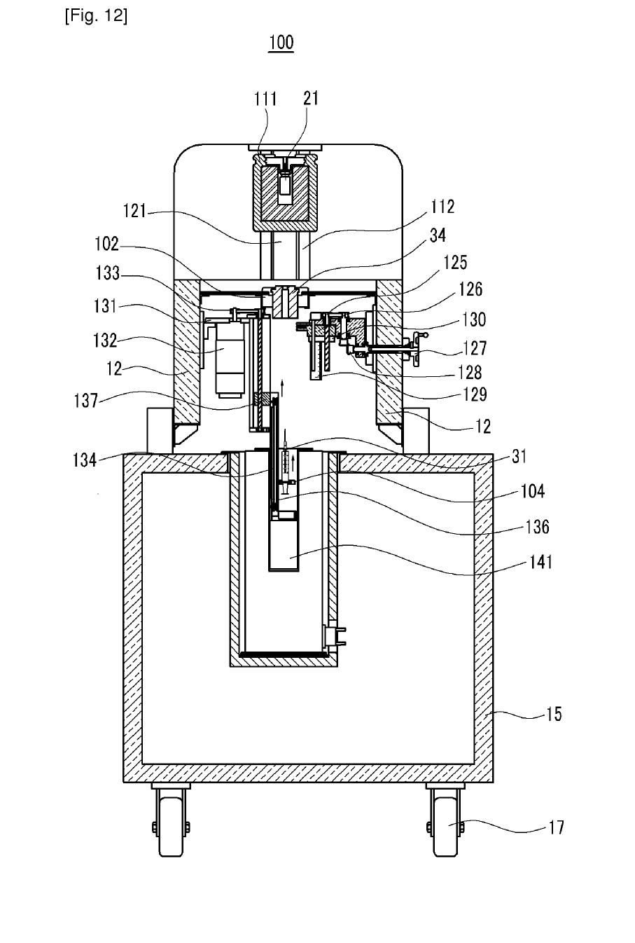

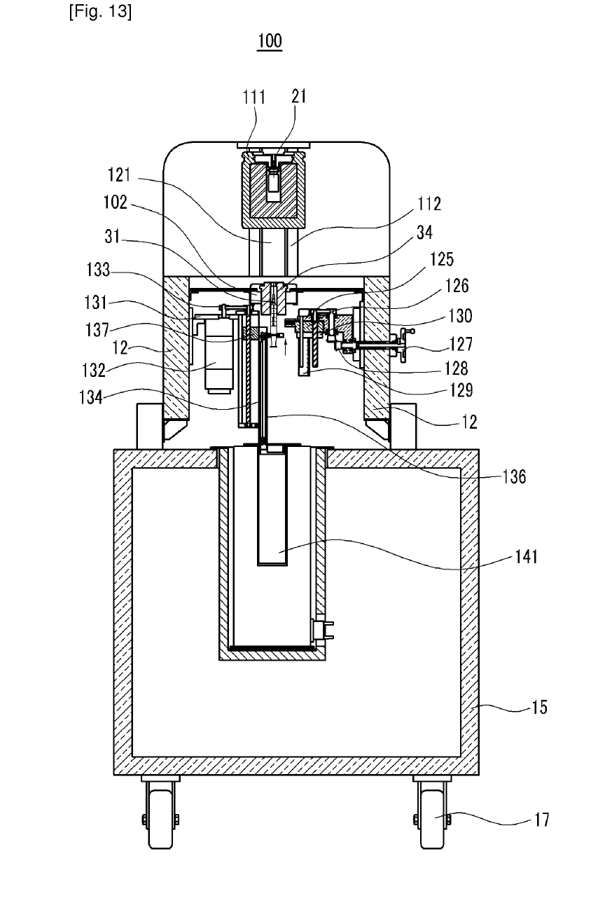

As shown in FIGS. 1 through 13, a system 100 according to a first embodiment of the present invention includes a radiopharmaceutical dispensing apparatus 101 and a radiation dosage measuring apparatus 141 which is integrally provided under the radiopharmaceutical dispensing apparatus 101. The radiopharmaceutical dispensing apparatus 101 dispenses a necessary dose of radiopharmaceuticals, contained in a vial 21, to an syringe 31, and a filling opening of the vial 21 is oriented upwards to prevent the remnant of the radiopharmaceuticals in the vial 21 from influencing measurement of radiation dosage. Thereafter, the syringe 31 into which radiopharmaceuticals is injected from the radiopharmaceutical dispensing apparatus 101 is directly moved downwards to the radiation dosage measuring apparatus 141 to measure the radiation dosage thereof. These processes can be continuously conducted.

The radiation dosage measuring apparatus 141 is installed in a cabinet 15 which is openable on opposite sides thereof. To facilitate movement of the entire system, a grip 16 is provided at a predetermined position on the upper surface of the cabinet 15, and wheels 17 are provided under the cabinet 15.

In the system 100 according to the first embodiment of the present invention, data on the dose of radiopharmaceuticals injected from the vial 21 into the syringe 31 and a radiation dosage of the radiopharmaceuticals are transmitted to a separate device for monitoring the medical examination.

The radiopharmaceutical dispensing apparatus 101 has a shielding wall 12 which is made, for example, of lead, and is provided on the perimeter of the dispensing apparatus 101 to surround radiopharmaceuticals, thus preventing radiation leakage when dispensing radiopharmaceuticals.

Particularly, an syringe insert door 11 which is made, for example, of lead glass, is provided on the central portion of the front surface of the radiopharmaceutical dispensing apparatus 101 so as to be movable to one side, thus facilitating the insertion or removal of the syringe 31 into or from the radiopharmaceutical dispensing apparatus 101, and allowing an operator to observe the interior of the radiopharmaceutical dispensing apparatus 101. Furthermore, a control box 13 is provided on a predetermined portion of the front surface of the radiopharmaceutical dispensing apparatus 101 to control it.

In the radiopharmaceutical dispensing apparatus 101, the syringe 31 is inserted into a radiation shield 34 which is made of lead, tungsten, etc. The radiation shield 34 is supported by a radiation shield support 102 such that the radiation shield 34 is maintained in a state of being oriented upwards. The radiation shield support 102 is slidably provided in the central portion of the radiopharmaceutical dispensing apparatus 101. Hence, in the state in which the syringe insert door 11 is in the open state, the operator can insert the radiation shield 34 provided with the syringe 31 into a seating hole 103 of the radiation shield support 102 and position the syringe 31 by pushing the radiation shield support 102 to the center of the radiopharmaceutical dispensing apparatus 101, or he/she can easily extract the syringe 31 along with the radiation shield 34 from the radiopharmaceutical dispensing apparatus 101 by pulling the radiation shield support 102.

The seating hole 103 is formed through the central portion of the radiation shield support 102. Here, the upper end of the seating hole 103 has a diameter larger than that of the lower end thereof to have a double stepped shape, thus forming a stopper in the seating hole 103, so that the syringe 31 received in the radiation shield 34 passes through the seating hole 103, but the radiation shield 34 is maintained in a state of being stopped by the stopper. Furthermore, when the syringe 31 is positioned by pushing the radiation shield support 102 to the center, it is firmly held by an syringe holder 104 which is disposed below the radiation shield support 102.

The syringe 31 includes a cylinder 32 which has a stop rim and is provided with an injection needle on a first end thereof, and the piston 33 which is inserted into a second end of the cylinder 32 so as to be slidable relative to the cylinder 32 and has a stop rim.

In the first embodiment of the present invention, the stop rim of the cylinder 32 of the syringe 31 is inserted into and held by the syringe holder 104.

A vial receiving casing 111 is provided above the radiation shield support 102. The vial receiving casing 111 includes a cylindrical vial receiving part which has an open upper end to receive the vial 21 therein, and a cover which covers the vial receiving part and has at a central position thereof a hole, through which the injection needle of the syringe 31 supported by the radiation shield support 102 and the syringe holder 104 is stuck into the vial 21. The vial receiving casing 111 is made of radiation shielding material.

Furthermore, a bottle casing drive unit is provided behind the vial receiving casing 111. The bottle casing drive unit includes a drive force generating means, such as a typical motor, which rotates the vial receiving casing 111 to orient the filling opening of the vial 21 upwards or downwards or moves the vial receiving casing 111, which is in the rotated state along with the vial 21, upwards or downwards. The bottle casing drive unit includes a drive force transmission means using a typical pulley, gear or threaded transmission structure.

Here, a bottle casing connection rod 113 couples the center of the rear portion of the vial receiving casing 111 to the bottle casing drive unit so as to transmit drive force from the bottle casing drive unit to the vial receiving casing 111.

The bottle casing drive unit includes a frame 112, a connection box 114, a casing lift motor 115, a casing lift force transmission means 116, a casing rotating motor 117 and a casing rotating force transmission means 118. Thus, when the casing lift motor 115 provided in the lower end of the frame 112 is operated, a vertical screw shaft is rotated by the drive force generated from the casing lift motor 115. Then, depending on the rotating direction of the vertical screw shaft, the vial receiving casing 111 is moved upwards or downwards through the casing lift force transmission means 116 including the connection box 114 which is threaded over the vertical screw shaft. Furthermore, the drive force, generated from the casing rotating motor 117 provided under the lower end of the connection box 114, can rotate the vial receiving casing 111 via the casing rotating force transmission means 118, such as bevel gears, provided in the connection box 114.

In addition, a preset dose dispensing unit is provided below the right side of the radiation shield support 102. The preset dose dispensing unit functions to dispense a preset dose of radiopharmaceuticals from the vial 21 into the syringe 31.

The preset dose dispensing unit includes a piston coupling protrusion 121, a protrusion pushing force transmission means 122, a spring 124, a piston guide 125, a piston vertical moving member 126, an injection dose control handle 127, a handle rotating force transmission means 128, a graduated ruler 129 and a digital graduation indicator 130.

The piston coupling protrusion 121 is provided under the lower portion of the bottle casing connection rod 113, so that when the vial receiving casing 111 is moved downwards in a state in which the vial 21 is oriented downwards to dispense a predetermined dose of radiopharmaceuticals from the vial 21 into the syringe 31, the piston coupling protrusion 121 is moved along with the vial receiving casing 111 and transmits drive force to the protrusion pushing force transmission means 122 using a cam structure, thus moving the piston guide 125 and the piston vertical moving member 126 together to the left, that is, towards the syringe 31. The piston guide 125 is disposed right above the piston vertical moving member 126.

When the piston vertical moving member 126 moves to the syringe 31, the piston guide 125 surrounds the piston 133 of the syringe 31, and the stop rim of the piston 33 is simultaneously inserted into the piston vertical moving member 126.

Here, the piston vertical moving member 126 is coupled to the piston guide 125 by a threaded coupling method so as to be movable upwards or downwards below the piston guide 125. An insert slot into which the stop rim of the piston 33 is inserted is formed at a predetermined position in the piston vertical moving member 126. Thus, when the piston vertical moving member 126, along with the piston guide 125, moves towards the syringe 31, the stop rim of the piston 33 is inserted into the insert slot of the piston vertical moving member 126. In this state, the piston vertical moving member 126 moves the piston 33 upwards or downwards using drive force transmitted from the injection dose control handle 127 thereto through the handle rotating force transmission means 128, such as a bevel gear, a pulley, etc.

The injection dose control handle 127 is provided on the right outer surface of the shielding wall 12 and is rotated to inject a predetermined dose of radiopharmaceuticals into the syringe 31. The rotating force of the injection dose control handle 127 is transmitted to the piston vertical moving member 126 through the handle rotating force transmission means 128, thus moving the piston vertical moving member 126 downwards. Then, in the state in which the injection needle of the syringe 31 held by the radiation shield support 102 and the syringe holder 104 has been stuck into the filling opening of the vial 21, the piston 33 of the syringe 31 is moved downwards. Thereby, a predetermined dose of radiopharmaceuticals is dispensed from the vial 21 into the syringe 31.

Furthermore, the graduated ruler 129 is attached to the piston guide 125 so that the operator can check the degree to which the piston vertical moving member 126 has been moved downwards, using the graduations. To prevent an error which may result during reading the graduations of the graduated ruler 125, the digital graduation indicator 130 is coupled to the piston vertical moving member 126 and moved along with it, thus enabling the operator to exactly read the graduations of the graduated ruler 125.

Only when the piston coupling protrusion 121 is moved downwards and the piston coupling protrusion 121 is thus pushed by the bottle casing connection rod 113, does the preset dose dispensing unit maintain the pushed state in which the dispensing operation can be conducted. When the vial receiving casing 111 is moved upwards and the piston coupling protrusion 121 is thus released from the pushed state, the preset dose dispensing unit is returned to its original state by an elastic member, such as the spring 124.

After injection of radiopharmaceuticals into the syringe 31 has been completed, the casing lift motor 115 is rotated in a reverse direction to move the vial receiving casing 111 upwards. Thereafter, the casing rotating motor 117 is also rotated in a reverse direction to orient the filling opening of the vial 21 upwards. Then, measurement of the radiation dosage can be prevented from being affected by the radiopharmaceuticals remaining in the vial 21. At this time, the piston coupling protrusion 121 is simultaneously moved upwards by contraction of the spring 124 which has been expended, thus preventing it from interfering with the movement of the syringe 31.

Meanwhile, an syringe lift unit is provided below the left side of the radiation shield support 102. The syringe lift unit moves the syringe holder 104 holding the syringe 31 upwards or downwards to measure a radiation dosage of radiopharmaceuticals injected into the syringe 31.

The syringe lift unit includes a frame 131, an syringe lift motor 132, an syringe lift force transmission means 133, a slide rail 134, an syringe holder lift unit 136 and a connection member 137.

The syringe lift motor 132 which is mounted at a predetermined position to the frame 131 transmits drive force to the slide rail 134 through the syringe lift force transmission means 133 and thus moves the syringe 31 upwards or downwards to insert the syringe 31 into the radiation dosage measuring apparatus 141 or remove it therefrom.

The connection member 137 connects the syringe lift force transmission means 133, which includes a belt, a gear, a screw shaft or the like, to the slide rail 134. The slide rail 134 is coupled to the syringe holder 104.

The slide rail 134 moves upwards or downwards in the radiation dosage measuring apparatus 141 depending on a direction in which the syringe lift motor 132 rotates. The slide rail 134 has a double structure such that it is extendable using a slide rail guide bar. Furthermore, the slide rail 134 is coupled at the upper end thereof to the syringe holder 104.

The syringe holder 104 moves upwards or downwards along with the slide rail 134 that moves upwards or downwards using drive force transmitted from the syringe lift motor 132. Simultaneously, the syringe holder 104 is moved with respect to the slide rail 134 towards the upper end or the lower end of the slide rail 134 by the syringe holder lift unit 136 including a timing belt and a pulley.

Thanks to this structure, when the slide rail 134 moves downwards, the syringe 31 held by the syringe holder 104 can also be moved downwards along with the slide rail 134 and be further moved downwards with respect to the slide rail 134 by the operation of the syringe holder lift unit 135. Therefore, although the entire height of the system 100 of the present invention is relatively low, the syringe 31 can sufficiently reach the radiation dosage measuring apparatus 141 which is provided at the lower position.

Furthermore, while the radiation dosage of radiopharmaceuticals in the syringe 31 is measured in the radiation dosage measuring apparatus 141, the vial 21 maintains the state of being oriented such that the filling opening thereof faces upwards in the radiopharmaceutical dispensing apparatus 101. Hence, the measurement of the radiation dosage of the radiopharmaceuticals that are in the syringe 31 can be prevented from being affected by the radiopharmaceuticals remaining in the vial 21 after some radiopharmaceuticals have been dispensed to the syringe 31. Thereby, in the single system, the dispensing work and the measurement work can be sequentially conducted.

The radiation dosage measuring apparatus 141 has a chamber for measuring radioactivity. The syringe 31 which has been removed from the radiation shield 34 is inserted into the chamber to measure a radiation dosage of the radiopharmaceuticals that have been injected into the syringe 31. The measured data is transmitted to a separate printer and is thus used as an important reference material when monitoring the medical examination.

Furthermore, the present invention further includes a control box 13 which is a control unit for controlling the vial casing drive unit, the preset dose dispensing unit and the syringe lift unit such that after a predetermined dose of radiopharmaceuticals is dispensed to the syringe 31 from the vial 21, a radiation dosage of the radiopharmaceuticals dispensed to the syringe 31 can be measured without being affected by radioactivity generated from the radiopharmaceuticals remaining in the vial 21.

In detail, the control unit controls the vial casing drive unit such that the injection needle of the syringe 31 is stuck into the filling opening of the vial 21 by moving the vial receiving casing 111 downwards after rotating the vial receiving casing 111 such that the filling opening of the vial 21 is oriented downwards. As well, the control unit controls the vial drive unit such that the injection needle of the syringe 31 is removed from the vial 21 by moving the vial receiving casing 111 upwards and the filling opening of the vial 21 is oriented upwards by rotating the vial receiving casing 111, thus preventing the radiation dosage measurement of the radiation dosage measuring apparatus 141 from being affected by the radiopharmaceuticals remaining in the vial 21.

Furthermore, the control unit controls the syringe lift unit such that the syringe 31 is moved upwards or, after radiopharmaceuticals have been injected into the syringe 31, it is moved downwards so that the radiation dosage measuring apparatus 141 can measure the radiation dosage of the radiopharmaceuticals of the syringe 31.

Meanwhile, in the first embodiment of the present invention, a separate drive motor may be used to automatically manipulate the injection dose control handle 127 to move, upwards or downwards, the piston 33 of the syringe 31 that is held by the syringe holder 104. In this case, the control unit also controls the preset dose dispensing unit.

The radiopharmaceutical dispensing operation and the radiation dosage measurement operation of the system 100 according to the first embodiment of the present invention having the above-mentioned construction will be explained in detail with reference to the attached drawings.

First, in the radiopharmaceutical dispensing operation according to the first embodiment of the present invention, the vial that contains radiopharmaceuticals therein and is received in the radiation shield is oriented such that the filling opening thereof faces downwards, and a predetermined dose of radiopharmaceuticals is dispensed to the syringe that is received in the radiation shield and is oriented such that the injection needle faces upwards.

In detail, the vial 21 containing radiopharmaceuticals, for example, [18F]FDG, is inserted into the vial receiving casing 111. Thereafter, the vial receiving casing 111 is moved to an upper position by the operation of the vial casing drive unit under the control of the control unit.

Subsequently, the syringe insert door 11 provided in the front surface of the system is opened, and the syringe 31 received in the radiation shield 34 is inserted into the seating hole 103 of the radiation shield support 102 such that the radiation shield 34 is supported on the stepped portion of the radiation shield support 102. In addition, the cylinder 32 of the syringe 31 is held by the syringe holder 104 which is disposed below the radiation shield support 102.

Here, the stop rim of the cylinder 32 is maintained in the state of being held by the syringe holder 104, but the stop rim of the piston 33 is not yet held by the piston vertical moving member 126, that is, it is maintained in a state of being standing by ready to couple to the piston vertical moving member 126.

Thereafter, the syringe insert door 11 is closed to ensure radiation shielding. In this state, the vial casing drive unit rotates the vial receiving casing 111 under the control of the control unit, such that the filling opening of the vial 21 is oriented downwards. Subsequently, the vial casing drive unit moves the vial receiving casing 111 downwards. Then, the injection needle of the syringe 31 is stuck into the vial 21 that is received in the vial receiving casing 111.

Simultaneously, the vial casing connection rod 113 moves downwards and thus pushes the piston coupling protrusion 121. Then, the piston guide 125 and the piston vertical moving member 126 are moved to the left, that is, towards the syringe 31, by the operation of the protrusion pushing force transmission means 122 which is operated in conjunction with the piston coupling protrusion 121. At this time, the piston vertical moving member 126 holds the stop rim of the piston 33.

In the state in which the stop rim of the piston 33 is held by the piston vertical moving member 126, the stop rim of the piston 33 is moved downwards by manipulating the injection dose control handle 127, so that a predetermined dose of radiopharmaceuticals is dispensed from the vial 21 to the syringe 31.

Here, the dose of radiopharmaceuticals injected into the syringe 31 can be checked using the graduated ruler 125. To prevent an error which may result when reading the graduated ruler 125, the digital graduation indicator 126 is used, thus increasing the accuracy.

Subsequently, the vial receiving casing 111, which contains the vial 21 containing the radiopharmaceuticals remaining after a portion thereof has been dispensed to the syringe 31, is moved upwards under the control of the control unit. Thus, the vial 21 is removed from the syringe 31, and the piston coupling protrusion 121 is simultaneously released from the pushed state.

Then, the piston guide 125 and the piston vertical moving member 126 are moved to the right by the restoring force of the spring 124 and are thus returned to their original positions, thereby releasing the stop rim of the piston 33 which has been held by the piston vertical moving member 126.

Thereafter, the vial receiving casing 111 is rotated under the control of the control unit such that the filling opening of the vial 21 is oriented upwards, thus reliably preventing the radiation dosage measurement of the radiation dosage measuring apparatus 141 from being affected by the radiopharmaceuticals remaining in the vial receiving casing 111.

Furthermore, the injection dose control handle 127 is rotated in the reverse direction so that the piston vertical moving member 126 is moved upwards by the drive force transmitted through the handle rotating force transmission means 128 and is maintained in the state of being coupled to the piston guide 125.

Next, in the process of measuring a radiation dosage of radiopharmaceuticals injected into the syringe according to the first embodiment of the present invention, the filling opening of the vial is oriented upwards such that the radiation dosage measurement is prevented from being affected by the radiopharmaceuticals remaining in the vial after some portion thereof has been injected into the syringe. In this state, the syringe containing the dispensed radiopharmaceuticals therein is moved downwards into the radiation dosage measuring apparatus.

In detail, drive force generated by rotating the syringe lift motor 132 under the control of the control unit is transmitted to the connection member 137 and the slide rail 134 through the syringe lift force transmission means 133, thus moving them downwards. At this time, the syringe holder 104 which holds the stop rim of the cylinder 32 of the syringe 31 is moved downwards along with the slide rail 134.

Simultaneously, the syringe holder lift unit 136 is operated by drive force transmitted through the syringe lift force transmission means 133, so that the syringe holder 104 is moved from the upper end of the slide rail 134 to the lower end thereof.

After a radiation dosage of radiopharmaceuticals in the syringe 31 has been measured in the state in which the syringe 31 is disposed in the radiation dosage measuring apparatus 141 by the operation of the syringe lift unit, in order to move the syringe upwards, the syringe lift unit is operated under the control of the control unit in the upwards direction, the reverse of that when moving the syringe 31 downwards.

Finally, after the radiopharmaceutical dispensing operation and the radiation dosage measurement operation have been completed, the operator opens the syringe insert door 11 and pulls out the syringe 31 along with the radiation shield 34 from the radiation shield support 102. Thereafter, the syringe 31 is used for administration to a patient.

Meanwhile, a system 100′ according to a second embodiment of the present invention is constructed such that a radiopharmaceutical dispensing apparatus 101 is integrated with a radiation dosage measuring apparatus 141 which is disposed under the radiopharmaceutical dispensing apparatus 101. In the same manner as the system 100 according to the first embodiment, a predetermined dose of radiopharmaceuticals contained in a vial 21 is dispensed to an syringe 31 by the radiopharmaceutical dispensing apparatus 101, and the syringe 31 containing radiopharmaceuticals dispensed by the radiopharmaceutical dispensing apparatus 101 is moved downwards into the radiation dosage measuring apparatus 141, and a radiation dosage of the radiopharmaceuticals in the syringe 31 is measured in the radiation dosage measuring apparatus 141 while the radiation dosage measurement is prevented from being affected by the radiopharmaceuticals remaining in the vial 21. In addition, these processes can be sequentially conducted in the same manner as that of the system 100 according to the first embodiment.

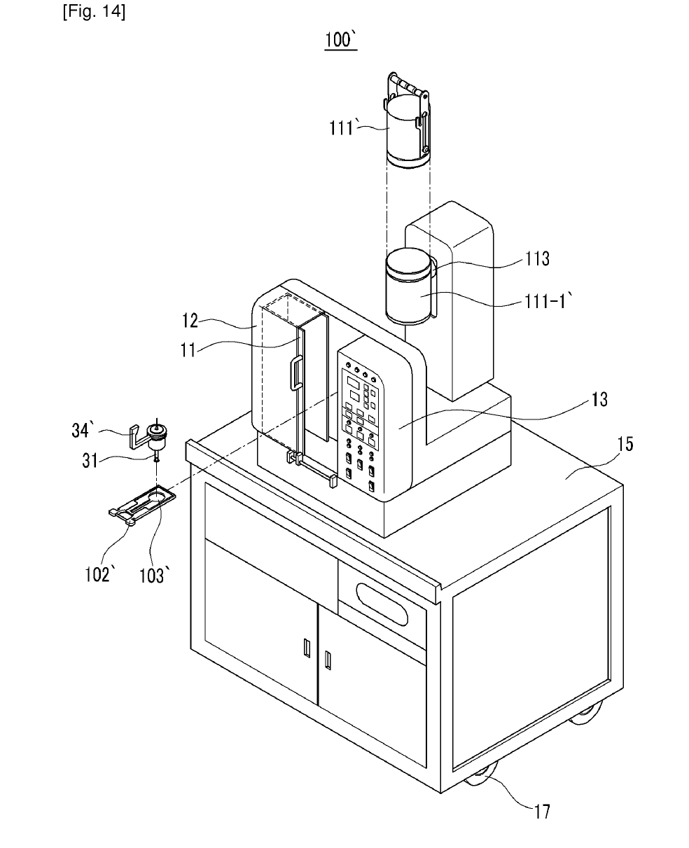

However, unlike the first embodiment, in the system 100′ according to the second embodiment, as shown in FIGS. 14 through 18, when a predetermined dose of radiopharmaceuticals is dispensed to the syringe 31, a process of moving a stop rim of a piston 33 of the syringe 31 upwards or downwards is automatized in such a way that drive force generated from an injection dose control motor 127′ is transmitted to the stop rim of the piston 33 through a dose control force transmission means 128′, thus facilitating the manipulation of the system 100′. Furthermore, a vial receiving casing 111′ including a casing receptacle 111-1′ can be moved only upwards, downwards, leftwards and rightwards but cannot be rotated, so that the radiation dosage measurement can be prevented from being affected by the radiopharmaceuticals remaining in the vial 21, and a position of a filling opening of the vial 21 can be maintained constant such that an injection needle of the syringe 31 can be exactly stuck into the filling opening of the vial 21. The following explanation of the second embodiment is focused on these, and the same explanation as that of the system 100 according to the first embodiment is deemed unnecessary.

As shown in FIG. 14, in the system 100′ according to the second embodiment of the present invention, the vial receiving casing 111′, which receives the vial 21 containing radiopharmaceuticals therein, is inserted in the casing receptacle 111-1′ such that the filling opening of the vial 21 is oriented downwards. Thereafter, the casing receptacle 111-1′ is moved downwards, so that the injection needle of the syringe 31 is stuck into the filling opening of the vial 21 through a needle passing hole which is formed through the casing receptacle 111-1′ at a position corresponding to the filling opening of the vial 21. Subsequently, radiopharmaceuticals are dispensed to, that is, inserted into, the syringe 31.

Meanwhile, a radiation shield 34′ has on a predetermined portion thereof a handle for facilitating the handling thereof. Furthermore, a seating hole 103′ is formed through a radiation shield support 102′ to receive the radiation shield 34′ therein. Thus, the syringe 31 is received in the radiation shield 34′, and the radiation shield 34′ is removably seated into the radiation shield support 102′ through the seating hole 103′.

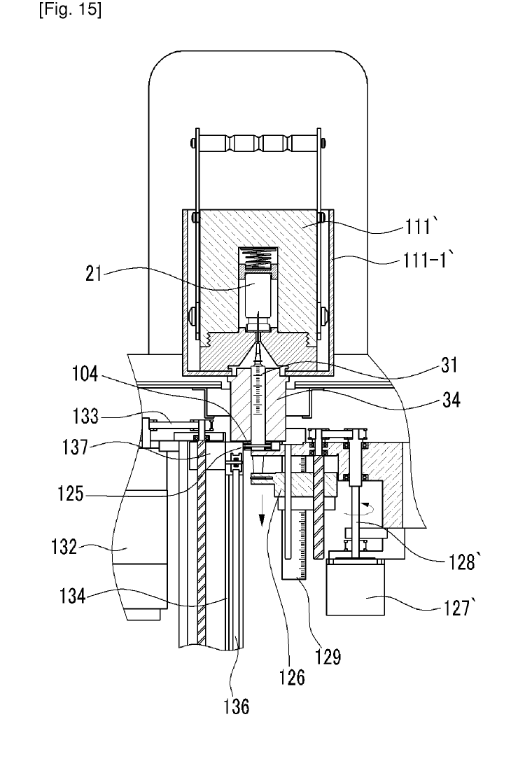

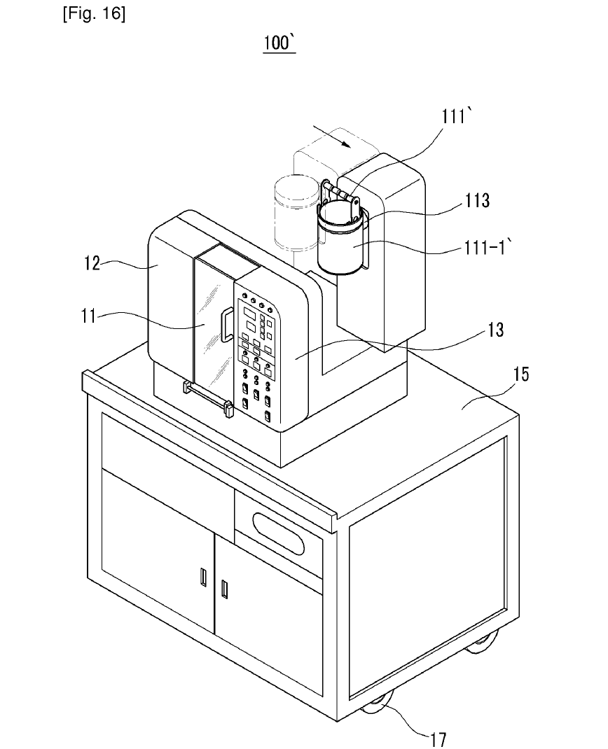

Moreover, as shown in FIGS. 15 and 16, the system 100′ is characterized in that when a necessary dose of radiopharmaceuticals is set, radiopharmaceuticals contained in the vial can be automatically dispensed to the syringe depending on the set dose.

To achieve the above purpose, unlike the system 100 of the first embodiment in which a dose of radiopharmaceuticals injected into the syringe 31 is manually set by rotating the injection dose control handle 127, in the second embodiment, a necessary dose of radiopharmaceuticals is set under the control of the control unit, and the stop rim of the piston 33 of the syringe 31 which is held by the piston vertical moving member 126 is moved downwards by drive force transmitted from the injection dose control motor 127′ through the dose control force transmission means 128′ depending on the set dose. Thereby, a necessary dose of radiopharmaceuticals can be automatically and precisely injected into the syringe 31.

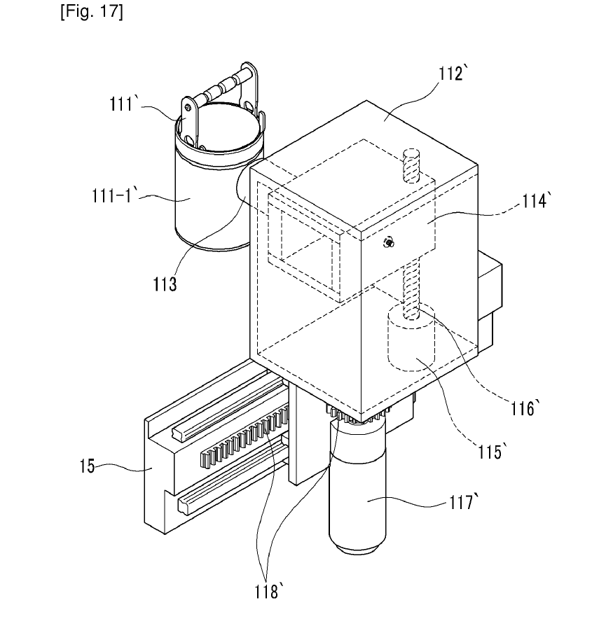

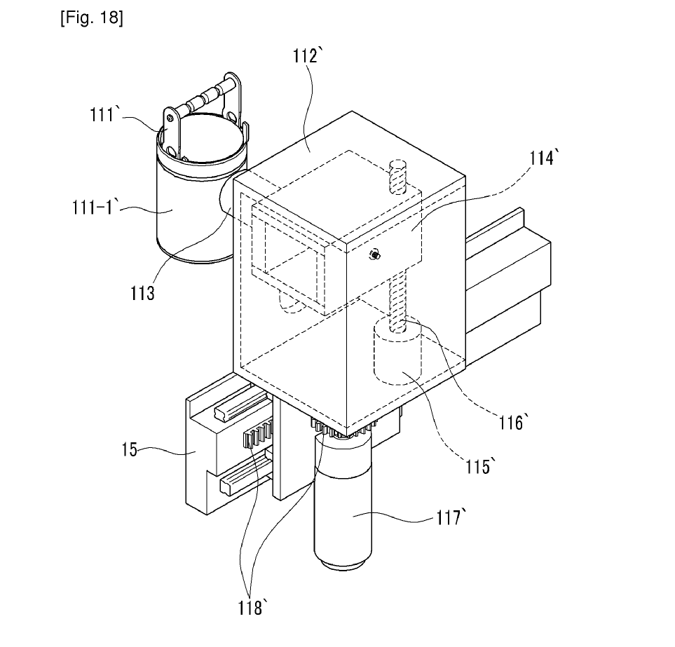

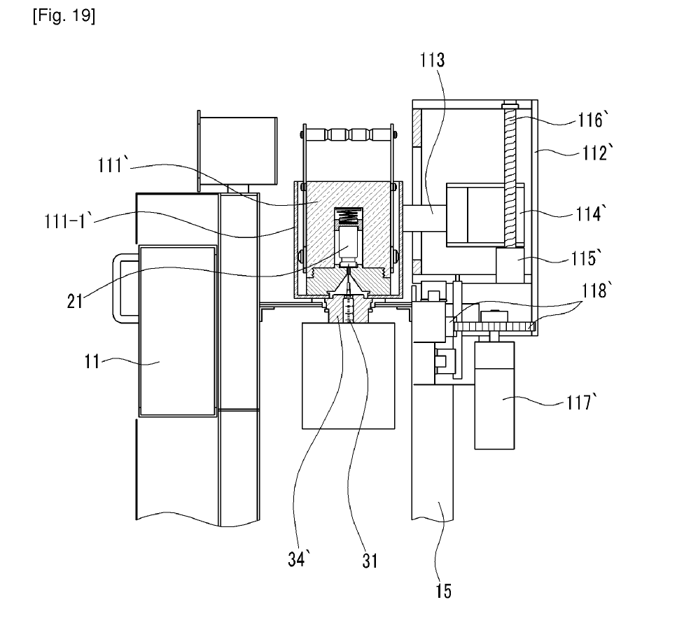

Furthermore, as shown in FIGS. 17 and 18, the system 100′ according to the second embodiment of the present invention is constructed such that when a radiation dosage of radiopharmaceuticals injected into the syringe is measured, the vial receiving casing can be moved upwards, downwards, leftwards or rightwards.

For this, a vial casing drive unit is connected to a casing receptacle 111-1′ through a vial casing connection rod 113. The vial casing drive unit includes a frame 112′, a connection box 114′, a casing lift motor 115′, a casing lift force transmission means 116′, a casing lateral drive motor 117′ and a casing lateral drive force transmission means 118′.

In the vial casing drive unit, the casing receptacle 111-1′ is fastened to the connection box 114′ through the vial casing connection rod 113. The connection box 114′ is threaded over a casing lift force transmission means 116′, such as a screw shaft. Drive force generated from the casing lift motor 115′ is transmitted to the casing lift force transmission means 116′. Then, the connection box 114′ moves along the casing lift force transmission means 116′ upwards or downwards depending on the rotating direction of the casing lift force transmission means 116′, thus moving the casing receptacle 111-1′ which contains the vial receiving casing 111′ upwards or downwards.

In addition, in the vial casing drive unit, the casing rotating motor 117′ which is provided on the lower portion of the frame 112′ generates drive force. The drive force of the casing rotating motor 117′ is transmitted to the casing receptacle 111-1′ through the casing lateral drive force transmission means 118′, such as pinion-rack gears. Thereby, the casing receptacle 111-1′ containing the vial receiving casing 111′ is moved leftwards or rightwards.

In the casing lateral drive force transmission means 118′, the pinion gear is provided on the frame 112′. The entire frame 112′ is moved by the rotation of the pinion gear, thus moving the casing receptacle 111-1′ leftwards or rightwards. The rack gear is fastened to the rear surface of the cabinet 15 at a position corresponding to the pinion gear.

Therefore, after the injection of radiopharmaceuticals into the syringe 31 has been completed under the control of the control unit, the casing receptacle 111-1′ is moved upwards by rotating the casing lift motor 115 in the reverse direction. Thereafter, unlike the system 100 according to the first embodiment in which the filling opening of the vial 21 is oriented upwards by rotating the casing rotating motor 117, the casing receptacle 111-1′ is moved rightwards to prevent the radiation dosage measurement from be affected by the radiopharmaceuticals remaining in the vial 21. Furthermore, thanks to this structure, the system 100′ of the second embodiment can avoid a risk in which the vial receiving casing 111′ is undesirably displaced from the correct position attributable to repeated rotation of the vial receiving casing 111′ when a predetermined dose of radiopharmaceuticals is dispensed from the vial 21 to the syringe 31. Therefore, the injection needle of the syringe 31 can be exactly stuck into the filling opening of the vial 21.

The radiopharmaceutical dispensing operation and the radiation dosage measurement operation of the system 100′ according to the second embodiment of the present invention having the above-mentioned construction will be explained in detail with reference to the attached drawings.

The radiopharmaceutical dispensing operation and the radiation dosage measurement operation according to the second embodiment are automatically conducted in the state in which the filling opening of the vial that is received in the radiation shield and contains radiopharmaceuticals is always oriented downwards.

First, in the radiopharmaceutical dispensing operation according to the second embodiment, the vial 21 containing radiopharmaceuticals therein is inserted into the vial receiving casing 111′, and the vial receiving casing 111′ is received into the casing receptacle 111-1′ such that the filling opening of the vial 21 is oriented downwards.

Thereafter, the syringe insert door 11 provided in the front surface of the system is opened. The radiation shield 34′ which is provided with the handle and contains the syringe 31 is supported by the radiation shield support 102. Subsequently, the radiation shield support 102 is pushed into the syringe insert door 11. Then, the cylinder 32 of the syringe 31 is held by the syringe holder 104 which is disposed below the radiation shield support 102.

Here, the stop rim of the cylinder 32 is maintained in the state of being held by the syringe holder 104, but the stop rim of the piston 33 is not yet held by the piston vertical moving member 126, that is, it is maintained in a state of being standing by ready to couple to the piston vertical moving member 126.

Thereafter, the syringe insert door 11 is closed to ensure radiation shielding. In this state, a necessary dose of radiopharmaceuticals is set under the control of the control unit. Then, the continuing radiopharmaceutical dispensing operation and the radiation dosage measurement operation are automatically performed.

In detail, when a necessary dose of radiopharmaceuticals is set under the control of the control unit, the casing receptacle 111-1′ is moved downwards by the operation of the vial casing drive unit. Thereby, the injection needle of the syringe 31 is stuck into the vial 21 that is received in the vial receiving casing 111′.

Simultaneously, the vial casing connection rod 113 moves downwards and thus pushes the piston coupling protrusion 121. Then, the piston guide 125 and the piston vertical moving member 126 are moved to the left, that is, towards the syringe 31, by the operation of the protrusion pushing force transmission means 122 which is operated in conjunction with the piston coupling protrusion 121. At this time, the piston vertical moving member 126 holds the stop rim of the piston 33.

In the state in which the stop rim of the piston 33 is held by the piston vertical moving member 126, the injection dose control motor 127′ is operated under the control of the control unit. Thus, the stop rim of the piston 33 is moved downwards by the drive force transmitted from the injection dose control motor 127′ through the dose control force transmission means 128′, so that a preset dose of radiopharmaceuticals is dispensed from the vial 21 to the syringe 31.

Subsequently, the casing receptacle 111-1′, which has therein the vial receiving casing 111′ including the vial 21 containing the remaining remnant of the radiopharmaceuticals after some portion thereof has been dispensed to the syringe 31, is moved upwards under the control of the control unit. Thus, the vial 21 is removed from the syringe 31, and the piston coupling protrusion 121 is simultaneously released from the pushed state. Then, the piston guide 125 and the piston vertical moving member 126 are returned to their original positions by the restoring force of the spring 124, thus releasing the stop rim of the piston 33 which has been held by the piston vertical moving member 126.

Thereafter, the casing receptacle 111-1′, which contains the vial receiving casing 111′ such that the vial 21 is oriented downwards, is moved rightwards by the control unit. Then, the vial 21 is displaced from the position aligning with the syringe 31, thus reliably preventing the radiation dosage measurement of the radiation dosage measuring apparatus 141 from being affected by the radiopharmaceuticals remaining in the vial receiving casing 111′.

Furthermore, the injection dose control motor 127′ is operated by the control unit so that the piston vertical moving member 126 is moved upwards by the drive force transmitted through the dose control force transmission means 128′ and is thus maintained in the state of being coupled to the piston guide 125.

Meanwhile, the process of measuring a radiation dosage of radiopharmaceuticals injected into the syringe according to the second embodiment of the present invention is the same as that of the system 100 of the first embodiment, therefore the explanation thereof is deemed unnecessary.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

As described above, in a system for dispensing radiopharmaceuticals and measuring a radiation dosage thereof according to the present invention, operation of dispensing a necessary dose of radiopharmaceuticals from a vial to an syringe and operation of measuring a radiation dosage of the radiopharmaceuticals contained in the syringe without being affected by the radiopharmaceuticals remaining in the vial can be sequentially conducted in the single system by external manipulation. Therefore, the operation can be conveniently, smoothly and exactly performed. Furthermore, the present invention can prevent or minimize a risk of radiation exposure.

1. A system for dispensing radiopharmaceuticals and measuring a radiation dosage thereof, comprising:
a radiopharmaceutical dispensing apparatus to dispense a predetermined dose of radiopharmaceuticals from a vial to an syringe, the vial being received in a first radiation shield and containing radiopharmaceuticals, the syringe being received in a second radiation shield; and
a radiation dosage measuring apparatus provided under the radiopharmaceutical dispensing apparatus to measure a radiation dosage of the radiopharmaceuticals dispensed to the syringe which is moved downwards from the radiopharmaceutical dispensing apparatus,
wherein when the radiation dosage measuring apparatus measures the radiation dosage of the radiopharmaceuticals in the syringe, a filling opening of the vial is disposed at a displaced position to prevent the radiation dosage measurement from being affected by radiopharmaceuticals remaining in the vial after the predetermined dose of radiopharmaceuticals is dispensed to the syringe.

2. The system according to claim 1, wherein the radiopharmaceutical dispensing apparatus comprises:
an syringe holder to support the syringe such that an injection needle of the syringe is oriented upwards;
a vial casing drive unit provided above the syringe holder to drive a vial receiving casing containing the vial therein;
a preset dose dispensing unit to dispense a preset dose of radiopharmaceuticals from the vial to the syringe by moving a piston of the syringe upwards or downwards in a state in which the injection needle of the syringe is stuck into the filling opening of the vial;
an syringe lift unit to move, upwards or downwards, the syringe holder supporting the syringe, into which the preset dose of radiopharmaceuticals is injected by the preset dose dispensing unit; and
a control unit to control the vial casing drive unit such that when the vial receiving casing is moved downwards while the vial receiving casing is oriented such that the filling opening of the vial faces downwards, the injection needle of the syringe supported by the syringe holder is stuck into the filling opening of the vial and the preset dose dispensing unit holds the piston of the syringe supported by the syringe holder, the control unit controlling the vial casing drive unit such that when the vial receiving casing is moved upwards, the injection needle of the syringe is removed from the vial and the preset dose dispensing unit releases the piston of the syringe, and then the vial receiving casing is moved such that the filling opening of the vial is oriented upwards, the control unit controlling the preset dose dispensing unit to move the piston of the syringe held by the preset dose dispensing unit upwards or downwards, the control unit controlling the syringe lift unit to move the syringe containing the preset dose of radiopharmaceuticals upwards or downwards.

3. The system according to claim 1, wherein the radiopharmaceutical dispensing apparatus comprises:
an syringe holder to support the syringe such that an injection needle of the syringe is oriented upwards;
a vial casing drive unit provided above the syringe holder to drive a vial receiving casing containing the vial therein;
a preset dose dispensing unit to dispense a preset dose of radiopharmaceuticals from the vial to the syringe by moving a piston of the syringe upwards or downwards in a state in which the injection needle of the syringe is stuck into the filling opening of the vial;
an syringe lift unit to move, upwards or downwards, the syringe holder supporting the syringe, into which the preset dose of radiopharmaceuticals is injected by the preset dose dispensing unit; and
a control unit to control the vial casing drive unit such that when the vial receiving casing is moved downwards while the vial receiving casing is oriented such that the filling opening of the vial faces downwards, the injection needle of the syringe supported by the syringe holder is stuck into the filling opening of the vial and the preset dose dispensing unit holds the piston of the syringe supported by the syringe holder, the control unit controlling the vial casing drive unit such that when the vial receiving casing is moved upwards, the injection needle of the syringe is removed from the vial and the preset dose dispensing unit releases the piston of the syringe, and then the vial receiving casing is moved leftwards or rightwards, the control unit controlling the preset dose dispensing unit to move the piston of the syringe held by the preset dose dispensing unit upwards or downwards, the control unit controlling the syringe lift unit to move the syringe containing the preset dose of radiopharmaceuticals upwards or downwards.
